(19) United States
(12) Patent Application Publication
(10) Pub. No.: US 2026/0026949 A1
(43) Pub. Date: Jan. 29, 2026

(54) HELICALLY BRAIDED ENDOPROSTHESIS

(71) Applicant: BOSTON SCIENTIFIC SCIMED, INC., Maple Grove, MN (US)

(72) Inventors: GENE THOMAS STORBECK, Millis, MA (US); TIMOTHY LAWRENCE RUBESCH, Blaine, MN (US); ANTHONY FRANK TASSONI, Jr., Andover, MN (US); MATTHEW MACIEJ, Rogers, MN (US)

(73) Assignee: BOSTON SCIENTIFIC SCIMED, INC., Maple Grove, MN (US)

(21) Appl. No.: 19/276,817

(22) Filed: Jul. 22, 2025

Related U.S. Application Data
(60) Provisional application No. 63/674,381, filed on Jul. 23, 2024.

(51) Int. Cl. A61F 2/90; D04C 1/06

(52) U.S. Cl. CPC A61F 2/90; D04C 1/06; A61F 2240/001; D10B 2509/00

(57) ABSTRACT

An endoprosthesis extending along a central longitudinal axis includes a plurality of filaments interwoven to form a plurality of closed cells having corners defined by cross-over points formed by the filaments. Each filament is oriented at a different angle relative to the central longitudinal axis. The cross-over points of each closed cell may include proximal and distal cross-over points, and first and second lateral cross-over points, wherein the distal cross-over point is circumferentially offset from the proximal cross-over point of its respective closed cell. A method of manufacturing includes securing a plurality of filaments extending from a braiding machine to a mandrel such that the filaments form a braid tent oriented at an intersecting angle with the mandrel; and interweaving the filaments around the mandrel to form a plurality of closed cells having corners defined by cross-over points formed by the filaments while rotating the mandrel.

### CROSS-REFERENCE TO RELATED APPLICATIONS

The application claims the benefit of U.S. Provisional Patent Application Ser. No. 63/674,381, filed on Jul. 23, 2024, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, renal tract, tracheal/bronchial tubes, and bile duct, as well as in a variety of other applications in the body.

Typical braided endoprostheses have a generally uniform and/or symmetrical construction, where each individual filament is oriented at the same angle relative to the central longitudinal axis of the endoprosthesis. This construction may provide mechanical and/or functional characteristics that are preferred or ideal in one respect, while compromising on others. Some properties considered when choosing an endoprosthesis for use may include axial foreshortening, radial expansion under pressure, radial compression or radial contraction under pressure (e.g., hoop strength), stiffness and/or flexibility, resistance to migration, axial strength under tension and/or compression, torqueability, etc. For example, a typical braid may be optimized to prevent axial foreshortening and/or prevent radial expansion under pressure. However, such a configuration may not be ideal for other properties that may be desired for a particular use.

In some instances, different endoprosthesis configurations may provide different combinations of foreshortening, deliverability, flexibility, conformability, radial force/strength, torqueability, and/or anchoring/migration characteristics. Of the known medical devices and methods, each has certain advantages and disadvantages. There is an ongoing need to provide alternative medical devices as well as alternative methods for manufacturing and using medical devices.

### SUMMARY

In one example, an endoprosthesis having a length extending along a central longitudinal axis from a proximal end to a distal end may comprise a plurality of filaments interwoven to form a plurality of closed cells extending along the length of the endoprosthesis. Each closed cell may have corners defined by cross-over points formed by the plurality of filaments. Each filament of the plurality of filaments forming a cross-over point of the plurality of cross-over points may be oriented at a different angle relative to the central longitudinal axis.

In addition, or alternatively, to any example disclosed herein, the plurality of filaments comprises at least a first filament extending in a first helical direction and a second filament extending in a second helical direction different from the first helical direction.

In addition, or alternatively, to any example disclosed herein, the first filament is oriented at a first angle relative to the central longitudinal axis as viewed perpendicular to the central longitudinal axis at the cross-over point and the second filament is oriented at a second angle relative to the central longitudinal axis as viewed perpendicular to the central longitudinal axis at the cross-over point, the first angle being different from the second angle.

In addition, or alternatively, to any example disclosed herein, the first angle and the second angle are interior angles within one closed cell of the plurality of closed cells.

In addition, or alternatively, to any example disclosed herein, the cross-over point is a proximal cross-over point of a closed cell and an imaginary line extending through the proximal cross-over point of the closed cell and a distal cross-over point of the closed cell as viewed perpendicular to the central longitudinal axis at the proximal cross-over point of the closed cell is oriented at a third angle relative to the central longitudinal axis, the third angle being different from the first angle and the second angle.

In addition, or alternatively, to any example disclosed herein, adjacent turns of the first filament are parallel to each other and adjacent turns of the second filament are parallel to each other.

In addition, or alternatively, to any example disclosed herein, proximal and distal corners of each closed cell, as viewed in a direction parallel to the central longitudinal axis, are offset circumferentially from each other.

In addition, or alternatively, to any example disclosed herein, opposing lateral corners of each closed cell, as viewed in a direction parallel to the central longitudinal axis, are offset longitudinally from each other.

In addition, or alternatively, to any example disclosed herein, the endoprosthesis may comprise at least one longitudinal reinforcing element interwoven within the plurality of filaments.

In addition, or alternatively, to any example disclosed herein, the at least one longitudinal reinforcing element extends helically around the central longitudinal axis.

In addition, or alternatively, to any example disclosed herein, in a second example, an endoprosthesis having a length extending along a central longitudinal axis from a proximal end to a distal end may comprise a plurality of filaments interwoven to form a plurality of closed cells extending along the length of the endoprosthesis. Each closed cell may have corners defined by cross-over points formed by the plurality of filaments, the cross-over points of each closed cell including a proximal cross-over point, a distal cross-over point, a first lateral cross-over point, and a second lateral cross-over point. The distal cross-over point of each closed cell may be circumferentially offset from the proximal cross-over point of its respective closed cell.

In addition, or alternatively, to any example disclosed herein, a first closed cell comprises a first proximal cross-over point and a first distal cross-over point and a second closed cell comprises a second proximal cross-over point and a second distal cross-over point.

In addition, or alternatively, to any example disclosed herein, the second proximal cross-over point is the first distal cross-over point.

In addition, or alternatively, to any example disclosed herein, the first proximal cross-over point, the first distal cross-over point, and the second distal cross-over point define an imaginary line oriented non-parallel to the central longitudinal axis.

In addition, or alternatively, to any example disclosed herein, the first proximal cross-over point, the first distal cross-over point, and the second distal cross-over point are aligned helically around the central longitudinal axis.

In addition, or alternatively, to any example disclosed herein, the plurality of filaments is configured to resist axial elongation of the endoprosthesis when a radially compressive force is applied to the endoprosthesis.

In addition, or alternatively, to any example disclosed herein, in a third example, a method of manufacturing an endoprosthesis may comprise: securing a plurality of filaments extending from a braiding machine to a mandrel such that the plurality of filaments forms a braid tent oriented at an intersecting angle with the mandrel; and interweaving the plurality of filaments around the mandrel to form a plurality of closed cells having corners defined by cross-over points formed by the plurality of filaments while rotating the mandrel around a central longitudinal axis of the mandrel.

In addition, or alternatively, to any example disclosed herein, each filament of the plurality of filaments forming a cross-over point is oriented at a different angle relative to the central longitudinal axis as viewed perpendicular to the central longitudinal axis at the cross-over point.

In addition, or alternatively, to any example disclosed herein, a first filament of the plurality of filaments extending in a first helical direction around the central longitudinal axis and a second filament of the plurality of filaments extending in a second helical direction around the central longitudinal axis intersect at the cross-over point such that the first filament is oriented at a first angle relative to the central longitudinal axis as viewed perpendicular to the central longitudinal axis at the cross-over point, and the second filament is oriented at a second angle relative to the central longitudinal axis as viewed perpendicular to the central longitudinal axis at the cross-over point, the first angle being different from the second angle.

In addition, or alternatively, to any example disclosed herein, interweaving the plurality of filaments around the mandrel comprises simultaneously rotating the mandrel relative to the braiding machine while translating the mandrel longitudinally relative to the braiding machine.

In addition, or alternatively, to any example disclosed herein, a first puller mechanism is disposed on a first side of the mandrel and is oriented at a first angle to the mandrel, and a second puller mechanism is disposed on a second side of the mandrel opposite the first side relative to the central longitudinal axis and is oriented at a second angle to the mandrel.

In addition, or alternatively, to any example disclosed herein, the first puller mechanism and the second puller mechanism cooperate to rotate the mandrel relative to the braiding machine while translating the mandrel longitudinally relative to the braiding machine.

In addition, or alternatively, to any example disclosed herein, a filament guide spaced apart from the braiding machine within the braid tent is configured to redirect the plurality of filaments extending from the braiding machine to the mandrel such that the intersecting angle with the mandrel is between 70 degrees and 90 degrees.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of a braiding machine manufacturing an endoprosthesis;

FIG. 2 illustrates selected aspects of a portion of a prior art endoprosthesis made according to prior art methods;

FIG. 3 illustrates selected aspects of a portion of an endoprosthesis in accordance with the disclosure;

FIG. 4 illustrates selected aspects of a portion of an endoprosthesis in accordance with the disclosure;

FIG. 5 illustrates selected aspects of a portion of an endoprosthesis in accordance with the disclosure;

FIG. 6 is a side view of a portion of a braiding machine manufacturing the endoprosthesis of FIGS. 3-5 in accordance with the disclosure;

FIG. 7 is a top view of a portion of a braiding machine manufacturing the endoprosthesis of FIGS. 3-5 in accordance with the disclosure;

FIG. 8 is a side view of a portion of a braiding machine of FIG. 7 manufacturing the endoprosthesis of FIGS. 3-5 in accordance with the disclosure;

FIG. 9 is a side view of a portion of the braiding machines of FIGS. 6-8 manufacturing the endoprosthesis of FIGS. 3-5 in accordance with the disclosure;

FIGS. 10A-10C illustrate migration of the prior art endoprosthesis of FIG. 2 when subjected to peristaltic motion/forces; and

FIGS. 11A-11C illustrate the endoprosthesis of FIGS. 3-5 in accordance with the disclosure when subjected to peristaltic motion/forces.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

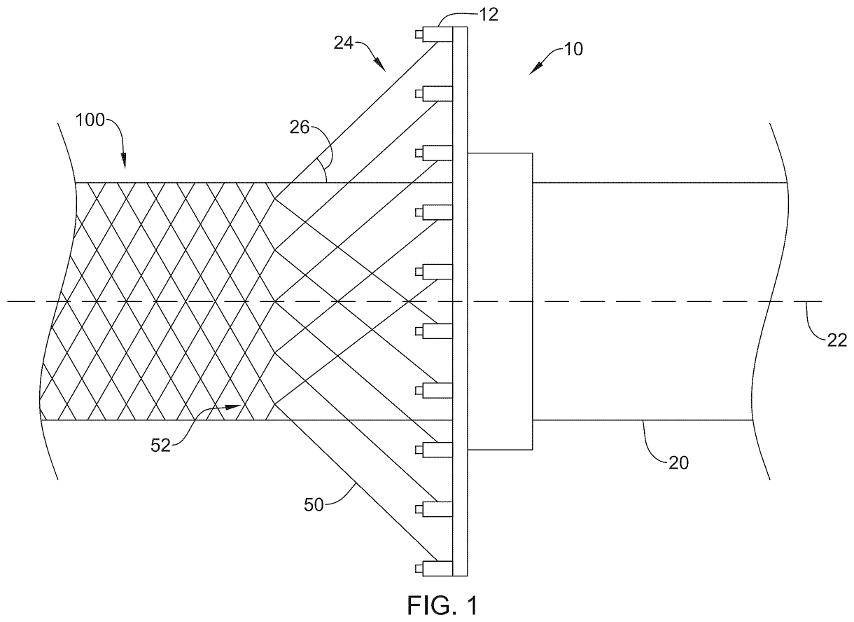

FIG. 1 is a side view illustrating selected aspects of a braiding machine 10. Some features and/or components of the braiding machine 10, such as motors, controls, safety features, etc. are not shown. However, the braiding machine 10 may include such features and/or components without limitation. In some embodiments, the braiding machine 10 may be a maypole braiding machine. For the purpose of the disclosure, the braiding machine 10 will be described in the context of the maypole braiding machine. However, in some embodiments, the braiding machine may be a rotary braiding machine, or some other type of braiding apparatus.

The braiding machine 10 comprises a plurality of notch gears. In some embodiments, each notch gear may comprise one or more braiding carriers 12 disposed thereon. In some embodiments, each notch gear may comprise a pair of braiding carriers 12 disposed thereon. In some embodiments, the one or more braiding carriers 12 may be disposed on alternating notch gears (e.g., on every other notch gear). The exact arrangement may be dependent upon the number of filaments used to construct the endoprosthesis. A mandrel 20 may extend from and/or through the braiding machine 10. The mandrel 20 may pass through the braiding machine 10 and is movable axially relative to the braiding machine 10. In some embodiments, the mandrel 20 may be omitted (e.g., the endoprosthesis may be constructed without the mandrel 20).

The plurality of notch gears may be arranged in a generally circular configuration around the mandrel 20 and/or a central axis of the braiding machine 10. Each notch gear may be configured to rotate around a center axis thereof, and each notch gear may be configured to rotate in an opposite direction to its neighboring and/or adjacent notch gear(s). As such, the plurality of notch gears may be configured to move in a counter-rotating manner that passes the one or more braiding carriers 12 in a sinusoidal fashion from one notch gear to an adjacent or juxtaposed notch gear, thereby causing the one or more braiding carriers 12 to revolve or move in a circumferential manner around the central axis of the braiding machine 10 around which the plurality of notch gears is arranged. In some embodiments, the central axis may be and/or may be coaxial with a central longitudinal axis 22 of the mandrel 20. The mandrel 20 is illustrated as having a round configuration (e.g., a circular cross-section), but the mandrel 20 is not necessarily restricted to the round configuration. In some instances, the mandrel 20 may have a square, triangular, etc. configuration or cross-section.

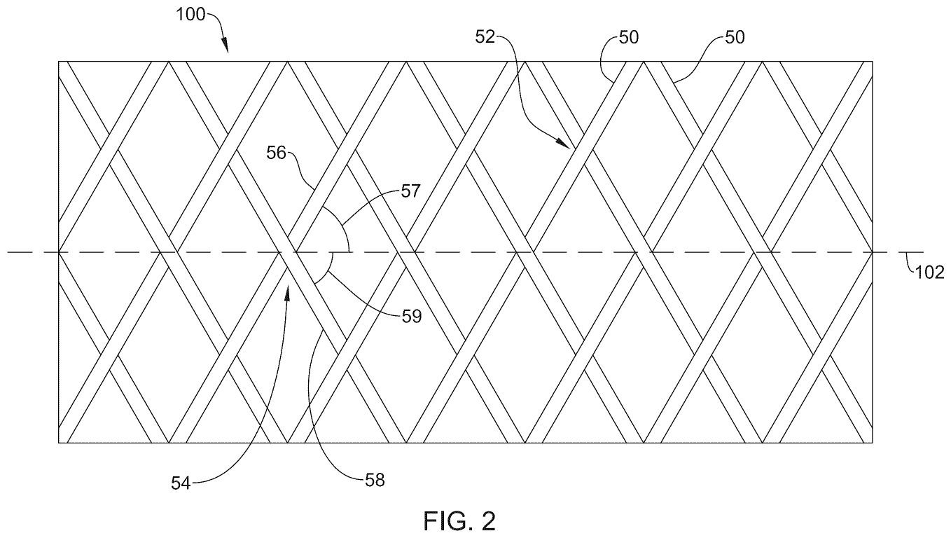

A plurality of filaments 50 may extend from the braiding machine 10 and/or the braiding carriers 12 to the mandrel 20, as is known in the art. The braiding machine 10 and/or the braiding carriers 12 may interweave (e.g., braid) the plurality of filaments 50 over and/or around the mandrel 20 to form an endoprosthesis 100, additional details of which are shown and discussed with respect to FIG. 2. The circular configuration of the plurality of notch gears and the one or more braiding carriers 12 achieves a generally circular but sinusoidal movement of the one or more braiding carriers 12 to interweave and/or braid the plurality of filaments 50 over and/or around the mandrel 20 to form the endoprosthesis 100.

In some embodiments, one filament of the plurality of filaments 50 may extend from each braiding carrier of the one or more braiding carriers 12. In some embodiments, more than one filament (e.g., 2 filaments, 3 filaments, 4 filaments, etc.) of the plurality of filaments 50 may extend from each braiding carrier of the one or more braiding carriers 12. In some embodiments, some braiding carriers of the one or more braiding carriers 12 may be devoid of the plurality of filaments 50. As such, the number of filaments used to make the endoprosthesis 100 may be varied and/or altered while using the same braiding machine 10.

During braiding, the mandrel 20 is moved axially along the central longitudinal axis in a controlled manner relative to the braiding machine 10. The one or more braiding carriers 12 revolve around the mandrel 20. The plurality of filaments 50 extend from the braiding machine 10 and/or the one or more braiding carriers 12 to the mandrel 20 to form a braid tent 24 having a generally conical configuration, as shown in FIG. 1. The braid tent and/or the plurality of filaments 50 may contact and/or engage with the mandrel 20 at an intersecting angle 26 with an outer surface of the mandrel 20. The intersecting angle 26 is typically between 35 and 65 degrees, and more commonly about 45 to 55 degrees.

An endoprosthesis produced by the braiding machine 10 of FIG. 1 has a uniform and/or constant arrangement along its length. The plurality of filaments 50 may be interwoven to form a plurality of closed cells. Each closed cell may have corners defined by cross-over points 52 formed by the plurality of filaments 50. The cross-over points 52 are aligned axially such that the cross-over points 52 are aligned substantially parallel to the central longitudinal axis 22 of the mandrel 20. In some cases, the mandrel 20 may be configured with securement projections (not shown) extending radially outwardly therefrom the ensure uniform alignment and/or spacing of the plurality of filaments 50 and/or the cross-over points 52, thereby producing the endoprosthesis 100, which may be representative of an endoprosthesis according to the prior art.

FIG. 2 is an enlarged, partial cutaway view illustrating selected aspects of the endoprosthesis 100 made according to prior art methods. As seen in FIG. 2, the endoprosthesis 100 may comprise a plurality of filaments 50 interwoven to define the cross-over points 52. A central longitudinal axis 102 of the endoprosthesis 100 is shown for reference. During manufacturing, the central longitudinal axis 102 of the endoprosthesis 100 may correspond to and/or may be coaxial with the central longitudinal axis 22 of the mandrel 20. Each filament of the plurality of filaments 50 is oriented at an angle relative to the central longitudinal axis 102 as viewed perpendicular to the central longitudinal axis 102 at a cross-over point 54. For ease of understanding, a first filament 56 and a second filament 58 defining the cross-over point 54 are identified and will be referenced herein. In the prior art endoprosthesis 100 formed according to prior art methods, the first filament 56 is oriented at a first angle 57 relative to the central longitudinal axis 102 as viewed perpendicular to the central longitudinal axis 102 at the cross-over point 54, and the second filament 58 is oriented at a second angle 59 relative to the central longitudinal axis 102 as viewed perpendicular to the central longitudinal axis 102 at the cross-over point 54. The first angle 57 and the second angle 59 are equal, thereby ensuring the cross-over points 52 are aligned axially along the length of the endoprosthesis 100. The first angle 57 and the second angle 59 may be modified and/or varied but are always the same relative to each other (e.g., if the first angle 57 is 55 degrees, the second angle 59 must be 55 degrees, etc.).

Generally, a high value for the first angle 57 and the second angle 59 (e.g., closer to 90 degrees) may produce more hoop strength and/or flexibility, while a low value for the first angle 57 and the second angle 59 (e.g., closer to zero degrees) may produce better tensile performance. Shifting the first angle 57 and the second angle 59 higher or lower to improve the associated characteristic(s) results in compromising the opposing characteristic(s). Designers and/or manufacturers often attempt to find a “middle ground” where the compromises are considered acceptable, but such compromises can result in some characteristics being less effective than would be desired.

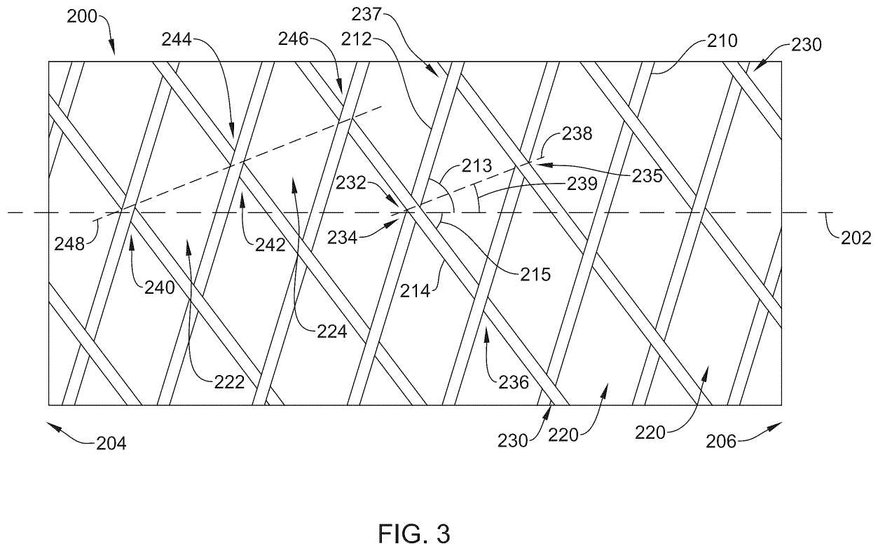

FIGS. 3-5 are enlarged, partial cutaway views illustrating selected aspects of an endoprosthesis 200 in accordance with the disclosure. In one example, the endoprosthesis 200 according to the disclosure may be configured to be positioned in a body lumen for a variety of medical applications. For example, the endoprosthesis 200 may be used to treat a stenosis in a blood vessel, may be used to maintain a fluid opening or pathway in the vascular, urinary, biliary, tracheobronchial, esophageal, gastrointestinal, or renal tracts, or may be used to position a device such as an artificial valve or a filter within a body lumen. In some instances, the endoprosthesis 200 may be a prosthetic graft, a stent-graft, or a stent (e.g., a vascular stent, tracheal stent, bronchial stent, esophageal stent, gastrointestinal stent, biliary stent, etc.). For the purposes of this disclosure, the terms “endoprosthesis” and “stent” may generally be used interchangeably. The endoprosthesis 200 may be any of a number of devices that may be introduced endoscopically, subcutaneously, percutaneously, or surgically to be positioned within an organ, tissue, or lumen, such as a heart, artery, vein, urethra, esophagus, trachea, bronchus, bile duct, or the like.

The endoprosthesis 200 may have a length extending along a central longitudinal axis 202 from a proximal end 204 to a distal end 206. While the proximal end 204 and distal end 206 are shown in the figures by reference number, it shall be understood that the figures do not necessarily show the entire length of the endoprosthesis 200 in the interest of clarity. As such, the proximal end 204 and distal end 206, which may be out of view in any given figure, are called out and/or designated for orientational and/or reference reasons. In some embodiments, the endoprosthesis 200 may comprise a plurality of filaments 210 interwoven to form a plurality of closed cells 220 extending along the length of the endoprosthesis 200. In some embodiments, each closed cell of the plurality of closed cells 220 may form a parallelogram shape rather than a rhombus shape (when seen in a flat pattern view), comprising four corners. In some embodiments, each closed cell of the plurality of closed cells 220 may have corners defined by cross-over points 230 formed by the plurality of filaments 210.

In some embodiments, the cross-over points 230 of each closed cell may include a proximal cross-over point 234, a distal cross-over point 235, a first lateral cross-over point 236, and a second lateral cross-over point 237, as shown in FIG. 3. In some embodiments, the proximal cross-over point 234 may be spaced apart longitudinally and/or axially from the distal cross-over point 235. In some embodiments, the first lateral cross-over point 236 may be spaced apart circumferentially and/or laterally from the second lateral cross-over point 237.

In some embodiments, proximal and distal corners of each closed cell (ref. 220), as viewed in a direction parallel to the central longitudinal axis 202, may be offset circumferentially from each other (e.g., to the left and/or right from each other as viewed in the direction parallel to the central longitudinal axis 202). In some embodiments, opposing lateral corners of each closed cell (ref. 220), as viewed in the direction parallel to the central longitudinal axis 202, may be offset longitudinally from each other (e.g., closer to and/or farther from the viewer).

In some embodiments, the plurality of filaments 210 may comprise at least a first filament 212 extending in a first helical direction around the central longitudinal axis 202 and a second filament 214 extending in a second helical direction around the central longitudinal axis 202 different from the first direction. In some embodiments, the plurality of filaments 210 may comprise multiple filaments (e.g., two, three, four, six, eight, ten, twelve, etc. filaments) ending in the first helical direction around the central longitudinal axis 202 and/or multiple filaments (e.g., two, three, four, six, eight, ten, twelve, etc. filaments) extending in the second helical direction around the central longitudinal axis 202. In some embodiments, the multiple filaments ending in the first helical direction around the central longitudinal axis 202 may be equal in number to the multiple filaments ending in the second helical direction around the central longitudinal axis 202. Other configurations are also contemplated.

In some embodiments, adjacent turns of the first filament 212 may be parallel to each other and adjacent turns of the second filament 214 may be parallel to each other. Accordingly, in at least some embodiments, adjacent turns of the first filament 212 may be equally spaced apart along the length of the endoprosthesis 200, and adjacent turns of the second filament 214 may be equally spaced apart along the length of the endoprosthesis 200.

The plurality of filaments 210 may be interwoven and/or braided around the central longitudinal axis 202 to form a braiding pattern. In some embodiments, the braiding pattern may comprise a one-over and one-under braiding pattern. In some embodiments, the braiding pattern may comprise a two-over and two-under braiding pattern. In some embodiments, the braiding pattern may comprise a one-over and one-under braiding pattern. In some embodiments, the braiding pattern may comprise pairs of filaments arranged in a one-over and one-under braiding pattern. some embodiments, the braiding pattern may comprise pairs of filaments arranged in a two-over and two-under braiding pattern. Other configurations and/or patterns are also contemplated.

In some embodiments, each filament of the plurality of filaments 210 forming a cross-over point 232 (e.g., at any selected cross-over point) of the plurality of cross-over points 230 may be oriented at a different angle relative to the central longitudinal axis 202 as viewed perpendicular to the central longitudinal axis 202 at the cross-over point 232 (e.g., at any selected cross-over point) of the cross-over points 230. In some embodiments, the first filament 212 may be oriented at a first angle 213 relative to the central longitudinal axis 202 as viewed perpendicular to the central longitudinal axis 202 at a cross-over point 232 (e.g., at any selected cross-over point) of the cross-over points 230 and the second filament 214 may be oriented at a second angle 215 relative to the central longitudinal axis 202 as viewed perpendicular to the central longitudinal axis 202 at the cross-over point 232, the first angle 213 being different from the second angle 215, as seen in FIG. 3. In some embodiments, an absolute value of the first angle 213 may be different from an absolute value of the second angle 215. In at least some embodiments, the first angle 213 may be an acute angle and/or the second angle 215 may be an acute angle.

In some embodiments, the first angle 213 and the second angle 215 may be interior angles within one closed cell of the plurality of closed cells 220. The one closed cell comprises and/or includes the cross-over point 232, since the cross-over point 232 is a point of reference for identifying the first angle 213 and the second angle 215. In some embodiments, the cross-over point 232 may be the proximal cross-over point 234 of a closed cell (ref. 220), and an imaginary line 238 extending through the proximal cross-over point 234 of the closed cell (ref. 220) and the distal cross-over point 235 of the closed cell (ref. 220) as viewed perpendicular to the central longitudinal axis 202 at the proximal cross-over point 234 of the closed cell (ref. 220) may be oriented at a third angle 239 relative to the central longitudinal axis 202. In at least some embodiments, the third angle 239 may be different from the first angle 213 and the second angle 215. In some embodiments, an absolute value of the third angle 239 may be different from the absolute value of the first angle 213 and the absolute value of the second angle 215. In at least some embodiments, the distal cross-over point 235 of each closed cell (ref. 220) may be circumferentially offset from the proximal cross-over point 234 of its respective closed cell (ref. 220).

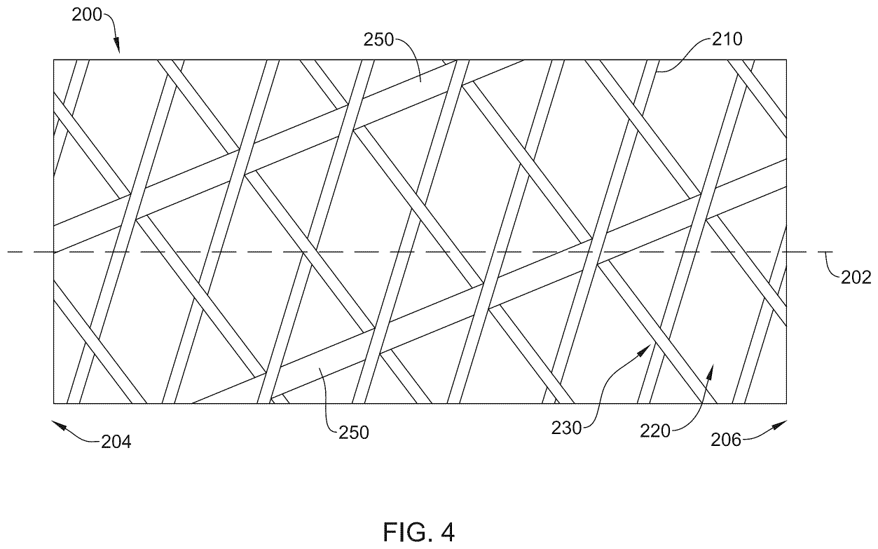

In some embodiments, a first closed cell 222 of the plurality of closed cells 220 may comprise a first proximal cross-over point 240 and a first distal cross-over point 242, and a second closed cell 224 of the plurality of closed cells 220 may comprise a second proximal cross-over point 244 and a second distal cross-over point 246. In at least some embodiments, the second proximal cross-over point 244 may be the first distal cross-over point 242. In some embodiments, the first proximal cross-over point 240, the first distal cross-over point 242, and the second distal cross-over point 246 may define an imaginary line 248 oriented non-parallel to the central longitudinal axis 202. In some embodiments, the first proximal cross-over point 240, the second proximal cross-over point 244, and the second distal cross-over point 246 may define the imaginary line 248 oriented non-parallel to the central longitudinal axis 202. In some embodiments, the first proximal cross-over point 240, the first distal cross-over point 242, the second proximal cross-over point 244, and the second distal cross-over point 246 may be aligned helically around the central longitudinal axis 202, as seen in FIG. 4.

Arranging the cross-over points 230 in a helical manner and/or forming the first angle 213 different from the second angle 215 may permit “tuning” and/or manipulating the properties of the endoprosthesis 200 to add or enhance additional properties to those present when the first angle 213 is equal to the second angle 215. In one example, an endoprosthesis 100 (e.g., FIG. 2) may be optimized for torqueability, wherein the first angle 57 and the second angle 59 may both be oriented at 45 degrees relative to the central longitudinal axis 102. However, by manufacturing the endoprosthesis 200 in accordance with the disclosure, the first angle 213 may be oriented at 45 degrees relative to the central longitudinal axis 202 to provide and/or improve torqueability and the second angle 215 may be oriented at 80-90 degrees relative to the central longitudinal axis 202 to provide and/or improve hoop strength, similar to properties found in a helical coil. Manufacturing the endoprosthesis 200 in the helical manner may produce properties of a braid and coil combination without the need for two separate structures and/or layers in the finished device, thereby reducing wall thickness, reducing stiffness, improving deliverability, and/or improving flexibility compared to a device having a braid and coil combination. As discussed herein, in some embodiments, the endoprosthesis 200 and/or the plurality of filaments 210 may be configured to resist axial elongation and/or migration of the endoprosthesis 200 when a radially compressive force is applied to the endoprosthesis 200 by a peristaltic lumen (e.g., the esophagus, the colon, etc.) because the helically arranged cross-over points 230 may cause the endoprosthesis 200 to “twist” while elongating as a radially compressive force is applied. However, engagement of the endoprosthesis 200 with the wall of the body lumen will resist the twisting motion of the endoprosthesis 200, thereby preventing radial compression and/or migration associated with peristaltic forces. Other configurations and/or combinations of properties are also contemplated.

In some embodiments, the endoprosthesis 200 may comprise at least one longitudinal reinforcing element 250 interwoven within the plurality of filaments 210, as seen in FIG. 4. In at least some embodiments, the at least one longitudinal reinforcing element 250 may extend helically around the central longitudinal axis 202. In some embodiments, the at least one longitudinal reinforcing element 250 may be oriented at a third angle relative to the central longitudinal axis 202 as viewed perpendicular to the central longitudinal axis 202. In at least some embodiments, the third angle may be different from the first angle 213 and/or the second angle 215 discussed above. In some embodiments, the at least one longitudinal reinforcing element 250 may improve resistance to axial tension and/or compression while permitting a desired level of flexibility and/or torqueability. In some embodiments, the at least one longitudinal reinforcing element 250 may act as and/or may provide properties found in or similar to helical coil, while the plurality of filaments 210 act as and/or may provide properties found in or similar to a braid.

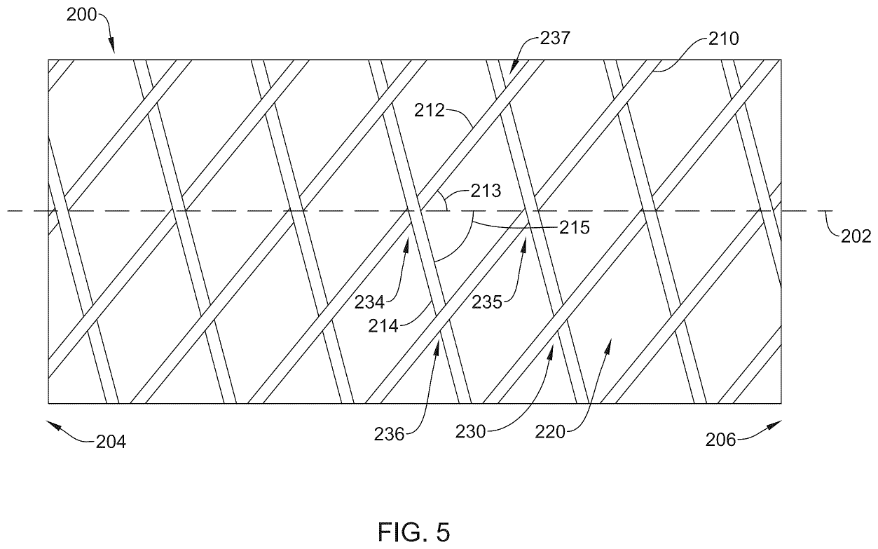

FIG. 5 illustrates selected aspects of an alternative configuration of the endoprosthesis 200. Except where noted, the endoprosthesis 200 may be configured and/or made as discussed above. In some embodiments, the endoprosthesis 200 may be formed such that the cross-over points 230 are aligned axially as viewed in the direction parallel to the central longitudinal axis 202 while each filament of the plurality of filaments 210 is oriented at a different angle relative to the central longitudinal axis 202 as viewed perpendicular to the central longitudinal axis 202 at a cross-over point 232 (e.g., at any selected cross-over point) of the cross-over points 230, as discussed above. In some embodiments, the endoprosthesis 200 may be formed such that the cross-over point 232 may be the proximal cross-over point 234 of a closed cell (ref. 220), and the imaginary line 238 extending through the proximal cross-over point 234 of the closed cell (ref. 220) and the distal cross-over point 235 of the closed cell (ref. 220) as viewed perpendicular to the central longitudinal axis 202 at the proximal cross-over point 234 of the closed cell (ref. 220) may be oriented parallel to the central longitudinal axis 202. In some embodiments, the alternative configuration shown in FIG. 5 may act and/or provide one or more properties similar to a conventional braid (e.g., the endoprosthesis 100) while permitting a different balance of properties provided by each filament of the plurality of filaments 210, as discussed herein (e.g., the endoprosthesis 200).

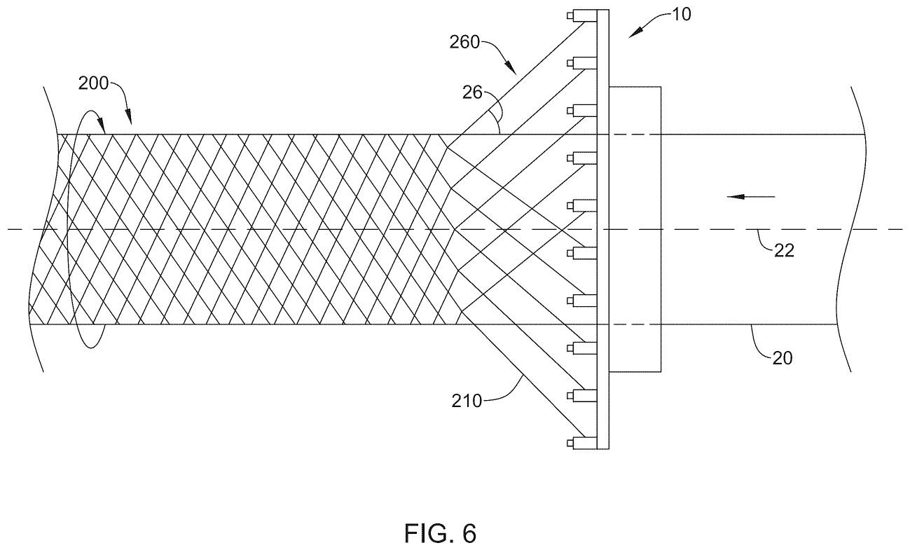

FIGS. 6-9 illustrate selected aspects related to an exemplary method of manufacturing the endoprosthesis 200. Other methods and apparatus are contemplated for use with the disclosure. In the interest of clarity, some aspects and/or elements may be shown in other figures in more detail. In some embodiments, the method may comprise securing a plurality of filaments 210 extending from a braiding machine 10 to a mandrel 20 such that the plurality of filaments 210 forms a braid tent 260 oriented at an intersecting angle 26 with an outer surface of the mandrel 20 and/or the central longitudinal axis 22 of the mandrel 20. Except where discussed otherwise, the braiding machine 10 may be constructed and/or arranged as discussed above.

In some embodiments, the method may comprise interweaving the plurality of filaments 210 around the mandrel 20 to form a plurality of closed cells 220 (e.g., FIG. 3) having corners defined by cross-over points 230 (e.g., FIG. 3) formed by the plurality of filaments 210 and/or at intersections of the plurality of filaments 210 while translating the mandrel 20 axially relative to the braiding machine 10. In some embodiments, the method may comprise interweaving the plurality of filaments 210 around the mandrel 20 to form a plurality of closed cells 220 having corners defined by cross-over points 230 formed by the plurality of filaments 210 and/or at intersections of the plurality of filaments 210 while rotating the endoprosthesis 200 and/or the mandrel 20 around the central longitudinal axis 22 of the mandrel 20, as seen in FIG. 6. In some embodiments, the mandrel 20 may be rotated directly via a motor, a servo, or other mechanical or non-automated means. In some embodiments, interweaving the plurality of filaments 210 around the mandrel 20 may comprise simultaneously rotating the endoprosthesis 200 and/or the mandrel 20 around the central longitudinal axis 22 of the mandrel 20 while translating the endoprosthesis 200 and/or the mandrel 20 axially relative to the braiding machine 10.

In some embodiments, some filaments of the plurality of filaments 210 may be introduced separately from the braiding carriers 12 (e.g., FIG. 1). In some embodiments, the at least one longitudinal reinforcing element 250 may be introduced into the endoprosthesis 200 separately from the plurality of filaments 210 and/or the braiding carriers 12 of the braiding machine 10. Other configurations are also contemplated.

In some embodiments, each filament of the plurality of filaments 210 forming a cross-over point 232 (e.g., FIG. 3) is oriented at a different angle relative to the central longitudinal axis 22 of the mandrel 20 and/or the central longitudinal axis 202 (e.g., FIG. 3) of the endoprosthesis 200 as viewed perpendicular to the central longitudinal axis (e.g., refs. 22, 202).

In some embodiments, a first filament 212 (e.g., FIG. 3) of the plurality of filaments 210 extending in a first helical direction around the central longitudinal axis (e.g., refs. 22, 202) and a second filament 214 (e.g., FIG. 3) of the plurality of filaments 210 extending in a second helical direction around the central longitudinal axis (e.g., refs. 22, 202) intersect at the cross-over point 232 such that the first filament 212 is oriented at a first angle 213 (e.g., FIG. 3) relative to the central longitudinal axis (e.g., refs. 22, 202) as viewed perpendicular to the central longitudinal axis (e.g., refs. 22, 202) at the cross-over point 232, and the second filament 214 is oriented at a second angle 215 (e.g., FIG. 3) relative to the central longitudinal axis (e.g., refs. 22, 202) as viewed perpendicular to the central longitudinal axis (e.g., refs. 22, 202) at the cross-over point 232. In at least some embodiments, the first angle 213 may be different from the second angle 215.

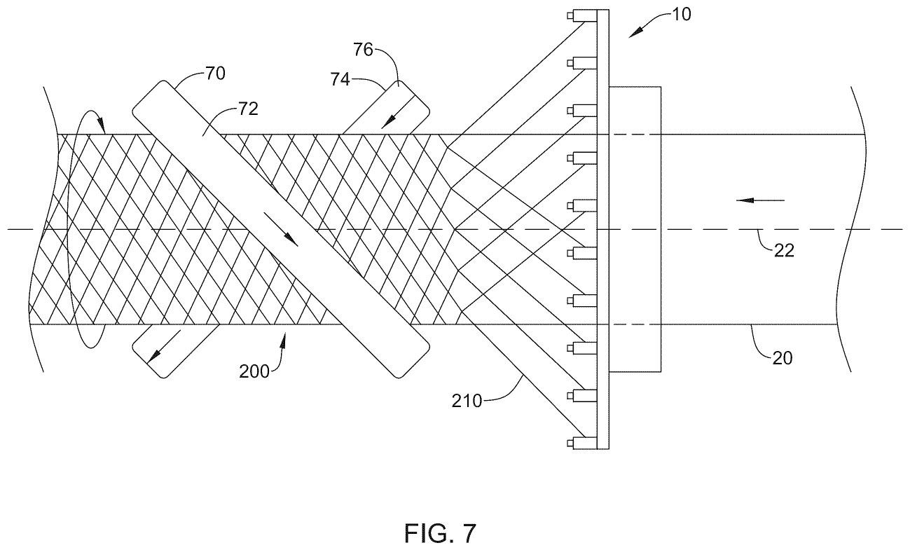

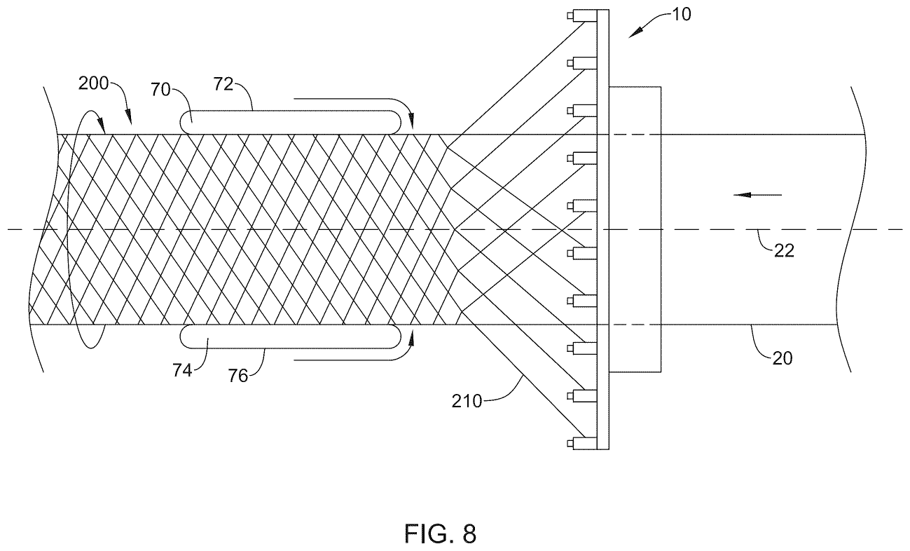

In some embodiments, a first puller mechanism 70 may be disposed on a first side of the mandrel 20 and may be oriented at a first puller angle to the mandrel 20 and/or the central longitudinal axis 22 of the mandrel 20, as seen in FIGS. 7-8. For reference, FIG. 7 schematically illustrates a top view and FIG. 8 schematically illustrates a side view. In some embodiments, the first puller mechanism 70 may comprise a drive motor (not shown) and a first drive belt 72 configured to rotate around a perimeter of the first puller mechanism 70. The arrow shown on the first drive belt 72 in FIG. 7 illustrates the direction of movement of the surface of the first drive belt 72 facing toward the viewer (e.g., out of the page). The opposing surface (facing into the page or away from the viewer) will move in the opposite direction. The arrow shown adjacent the first puller mechanism 70 in FIG. 8 illustrates the direction of movement of the first drive belt 72.

In some embodiments, a second puller mechanism 74 may be disposed on a second side of the mandrel 20 opposite the first side relative to the central longitudinal axis 22 of the mandrel 20 and may be oriented at a second puller angle to the mandrel 20 and/or the central longitudinal axis 22 of the mandrel 20, as seen in FIGS. 7-8. In some embodiments, the second puller mechanism 74 may comprise a drive motor (not shown) and a second drive belt 76 configured to rotate around a perimeter of the second puller mechanism 74. The arrow shown on the second drive belt 76 in FIG. 7 illustrates the direction of movement of the surface of the second drive belt 76 facing toward the viewer (e.g., out of the page). The opposing surface (facing into the page or away from the viewer) will move in the opposite direction. The arrow shown adjacent the second puller mechanism 74 in FIG. 8 illustrates the direction of movement of the second drive belt 76.

The second puller angle may be different from the first puller angle. In at least some embodiments, the first puller angle and/or the second puller angle may be non-parallel to the mandrel 20 and/or the central longitudinal axis 22. In some embodiments, the first puller angle and/or the second puller angle may be skewed with respect to the mandrel 20 and/or the central longitudinal axis 22.

In some embodiments, the drive motor of the second puller mechanism 74 may be a second drive motor distinct from the drive motor of the first puller mechanism 70. In some embodiments, a single drive motor may be operatively coupled to the first puller mechanism 70 and the second puller mechanism 74. Other configurations are also contemplated.

The first puller mechanism 70 and/or the first drive belt 72 may be configured to engage and/or may be engaged with the mandrel 20 and/or the endoprosthesis 200 disposed thereon. The second puller mechanism 74 and/or the second drive belt 76 may be configured to engage and/or may be engaged with the mandrel 20 and/or the endoprosthesis 200 disposed thereon.

In some embodiments, the first puller mechanism 70 and the second puller mechanism 74 may be configured to translate the endoprosthesis 200 and/or the mandrel 20 along the central longitudinal axis 22 relative to the braiding machine 10. In some embodiments, the first puller mechanism 70 and the second puller mechanism 74 may be configured to translate the endoprosthesis 200 and/or the mandrel 20 longitudinally relative to the braiding machine 10 along the central longitudinal axis 22. In some embodiments, the first puller mechanism 70 and the second puller mechanism 74 may be configured to translate the endoprosthesis 200 and/or the mandrel 20 longitudinally through and/or away from the braiding machine 10 along the central longitudinal axis 22.

In some embodiments, the first puller mechanism 70 and the second puller mechanism 74 may cooperate to rotate the endoprosthesis 200 and/or the mandrel 20 around the central longitudinal axis 22 and/or relative to the braiding machine 10 while translating the endoprosthesis 200 and/or the mandrel 20 longitudinally relative to the braiding machine 10, thereby causing the first filament 212 and/or the second filament 214 to wrap and/or extend helically around the mandrel 20 and/or the central longitudinal axis 22 as the plurality of filaments 210 is interwoven around the mandrel 20 to form the endoprosthesis 200.

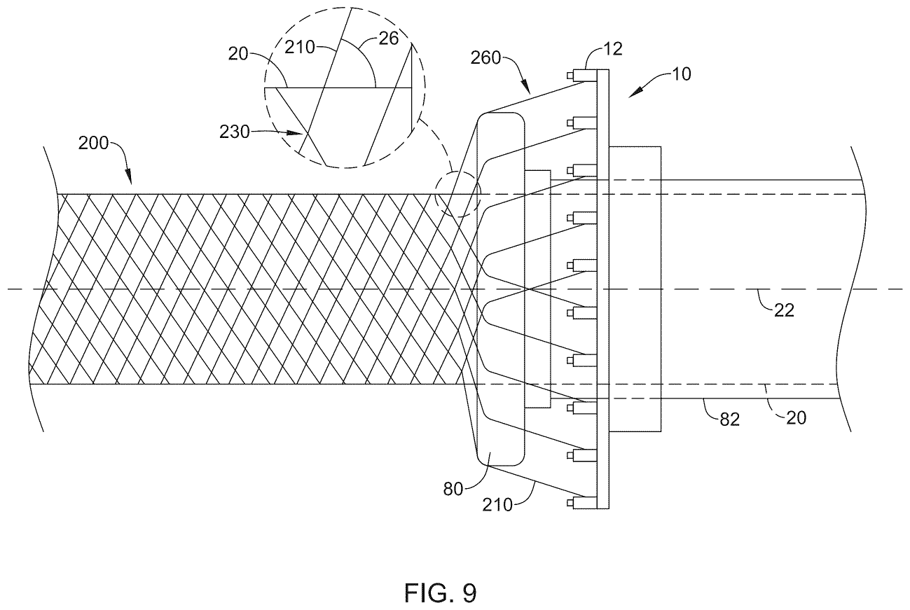

It is possible that when manufacturing the endoprosthesis 200, merely rotating the mandrel 20 while the intersecting angle 26 (e.g., FIG. 6) with the outer surface of the mandrel 20 and/or the central longitudinal axis 22 of the mandrel 20 is within a “normal” range (e.g., between 35 and 65 degrees) may produce an endoprosthesis 200 where the alignment and/or spacing of the plurality of filaments 210 and/or the cross-over points 230 is inconsistent. To reduce variation in the alignment and/or spacing of the plurality of filaments 210 and/or the cross-over points 230, and/or to ensure consistency in the alignment and/or spacing of the plurality of filaments 210 and/or the cross-over points 230, a filament guide 80 may be spaced apart from the braiding machine 10 along the central longitudinal axis 22 of the mandrel 20 within the braid tent 260, as shown in FIG. 9. The filament guide 80 may be configured to redirect the plurality of filaments 210 extending from the braiding machine 10 to the mandrel 20 such that the intersecting angle 26 with the outer surface of the mandrel 20 and/or the central longitudinal axis 22 of the mandrel 20 is between 70 degrees and 90 degrees. Accordingly, in at least some embodiments, the filament guide 80 may be configured to change the intersecting angle 26 (e.g., from a first intersecting angle corresponding to omission of the filament guide 80) to a second intersecting angle greater than the original intersecting angle 26 (e.g., the first intersecting angle). As the intersecting angle 26 and/or the second intersecting angle approaches 90 degrees, less variation and/or more consistency may be achieved in the alignment and/or spacing of the plurality of filaments 210 and/or the cross-over points 230 as the mandrel 20 is rotated relative to the braiding machine 10 while interweaving the plurality of filaments 210 around the mandrel 20 to produce the endoprosthesis 200.

In some embodiments, the filament guide 80 may be substantially circular and/or round in cross-sectional shape (taken perpendicular to the central axis of the braiding machine 10 and/or the central longitudinal axis 22 of the mandrel 20). The filament guide 80 may have a proximal face, a distal face, and a rounded outer perimeter and/or a rounded outer edge extending from the proximal face to the distal face. In at least some embodiments, the filament guide 80 may have a generally convex shape.

In some embodiments, a radial extent and/or an outer diameter of the filament guide 80 may be less than a radial extent and/or an outer diameter of the braiding machine 10. In one non-limiting example, the radial extent and/or the outer diameter of the filament guide 80 may be about 6 inches (about 15.24 centimeters). In other non-limiting examples, the radial extent and/or the outer diameter of the filament guide 80 may be about 4 inches (about 10.16 centimeters), 5 inches (about 12.7 centimeters), about 5.5 inches (about 13.97 centimeters), about 6.5 inches (about 16.51 centimeters), about 7 inches (about 17.78 centimeters), about 8 inches (about 20.32 centimeters), etc. Other configurations are also contemplated. In some embodiments, the plurality of notch gears and/or the one or more braiding carriers 12 may be disposed radially outward of the radial extent and/or the outer diameter of the filament guide 80, as measured perpendicular to the central axis of the braiding machine 10 and/or the central longitudinal axis 22 of the mandrel 20.

In some embodiments, the filament guide 80 may be constructed and/or configured such that the plurality of filaments 210 may slide along and/or over the filament guide 80 without damaging, cutting, snagging, abrading, stretching, etc. the plurality of filaments 210. In some embodiments, the filament guide 80 may be constructed and/or configured such that the plurality of filaments 210 may slide along and/or over the rounded outer perimeter and/or the rounded outer edge of the filament guide 80 without damaging, cutting, snagging, abrading, stretching, etc. the plurality of filaments 210. In some embodiments, the filament guide 80 may be constructed and/or configured such that the plurality of filaments 210 may slide along and/or over a convex surface of the filament guide 80 without damaging, cutting, snagging, abrading, stretching, etc. the plurality of filaments 210. In some embodiments, the filament guide 80 may be formed from a lubricious material. Some suitable but non-limiting examples of materials that may be used to form the filament guide 80, including polymeric materials, ceramic materials, composite materials, and the like, are discussed below. In some embodiments, the filament guide 80 may include a lubricious coating disposed thereon. Other configurations are also contemplated.

In at least some embodiments, the filament guide 80 may be mounted to a shaft 82, as seen in FIG. 9. The shaft 82 may extend from and/or through the braiding machine 10. The mandrel 20 may extend through and/or may pass through a lumen of the shaft 82. As such, the mandrel 20 may be movable relative to the shaft 82. In some embodiments, the shaft 82 and/or the filament guide 80 may be axially secured relative to the braiding machine 10 during operation of the braiding machine 10 (e.g., the filament guide 80 and/or the shaft 82 is not axially movable relative to the braiding machine 10 during operation). Axial spacing between the braiding machine 10 and/or one or more braiding carriers 12 and the filament guide 80 may correlate to and/or dictate the intersecting angle 26 and/or the second intersecting angle with the outer surface of the mandrel 20 and/or the central longitudinal axis 22 of the mandrel 20. Other configurations are also contemplated. The plurality of notch gears and/or the one or more braiding carriers 12 may be configured to rotate and/or revolve around the shaft 82 in the manner described herein with respect to the central axis of the braiding machine 10 and/or the central longitudinal axis 22 of the mandrel 20.

In some embodiments, the endoprosthesis 200 may be manufactured in discrete lengths, wherein the endoprosthesis 200 is made one at a time. In some embodiments, the endoprosthesis 200 may be manufactured in a continuous manner (e.g., one long, continuous structure) and cut to desired length(s) at a later time.

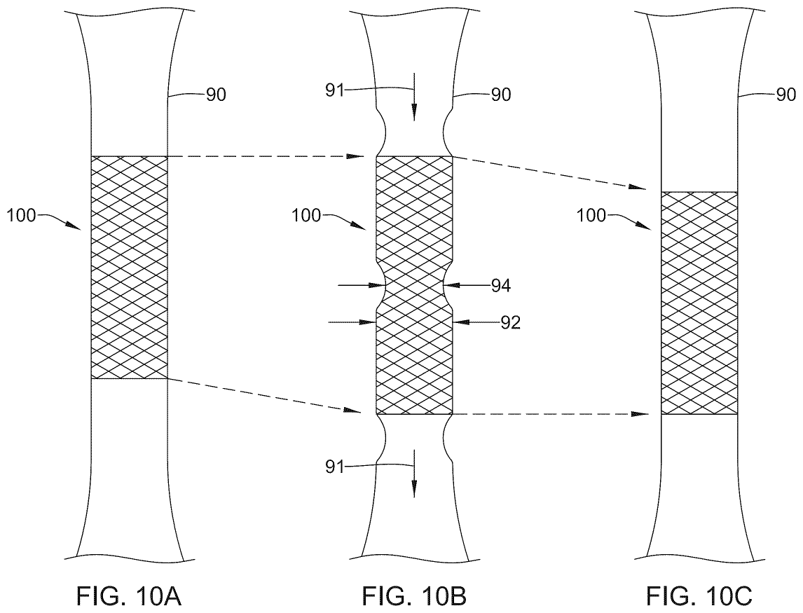

FIGS. 10A-C and 11A-C illustrate the effects of peristaltic motion and/or forces upon the prior art endoprosthesis 100 and the endoprosthesis 200 according to the current disclosure, respectively. As shown in FIG. 10A, the endoprosthesis 100 made according to prior art methods is disposed within a body lumen 90, which may be a peristaltic lumen such as the esophagus or the colon. The endoprosthesis 100 made according to prior art methods may be prone to and/or may suffer from migration under the normal peristaltic motion and/or forces present in the body lumen 90. A peristaltic wave 91 has a major diameter size 92 and a minor diameter size 94. The peristaltic wave 91, and/or the rhythmic forces of expansion and contraction caused by the peristaltic wave 91, moves particles, such as food or waste, downstream within the body lumen 90. The rhythmic forces of expansion and contraction caused by the peristaltic wave 91 are also directed to and/or onto the endoprosthesis 100 disposed within the body lumen 90. The endoprosthesis 100 made according to prior art methods, when subjected to the rhythmic forces of expansion and contraction caused by the peristaltic wave 91, similar to when the endoprosthesis 100 has a radially compressive force applied thereto when shifting towards a collapsed delivery configuration for delivery, expands axially and/or longitudinally (e.g., elongates), as shown in FIG. 10B. When the peristaltic wave 91, the rhythmic forces of expansion and contraction caused by the peristaltic wave 91, and/or the radially compressive force ends or is removed, the endoprosthesis 100 returns to its original and/or its expanded deployed configuration (e.g., its original and/or normal length in the expanded deployed configuration) having migrated downstream, as shown in FIG. 10C.

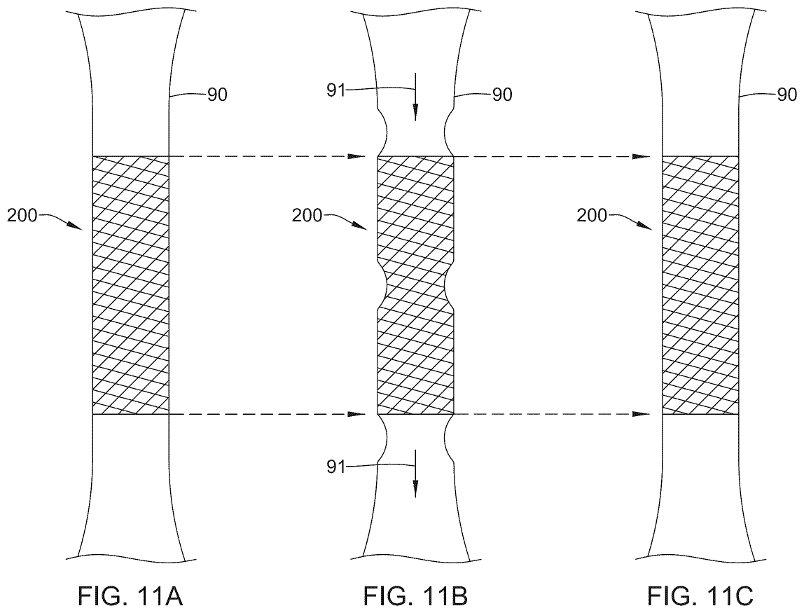

In some embodiments, downstream migration due to peristaltic motion and/or forces may be resisted and/or eliminated by constructing the endoprosthesis 200 according to the instant disclosure. FIG. 11A illustrates the endoprosthesis 200 made in accordance with the instant disclosure disposed within the body lumen 90. As the peristaltic wave 91, and/or the rhythmic forces of expansion and contraction caused by the peristaltic wave 91, is directed to and/or onto the endoprosthesis 200, as shown in FIG. 11B, the plurality of filaments 210 may be configured to resist axial elongation and/or migration. The endoprosthesis 200, in contrast to the endoprosthesis 100 described above, requires a third dimension and/or direction of force (e.g., a helical direction of force) in order to be radially compressed and/or reduced in size and thereby expanded (e.g., elongated) axially and/or longitudinally when subjected to the peristaltic wave 91, and/or the rhythmic forces of expansion and contraction caused by the peristaltic wave 91. The helical arrangement of the plurality of filaments 210 and/or the cross-over points 230 causes a twisting motion to be imparted onto the endoprosthesis 200 when a radially compressive force is applied to the endoprosthesis 200. Since the endoprosthesis 200 is engaged with the body lumen 90 in the expanded deployed configuration, the plurality of filaments 210 may be configured to resist axial elongation of the endoprosthesis 200 when the radially compressive force is applied to the endoprosthesis 200 by the peristaltic lumen (e.g., the body lumen 90) because the body lumen 90 resists the twisting motion and/or the helical direction of force required to expand (e.g., elongate) the endoprosthesis 200 axially and/or longitudinally. Accordingly, the endoprosthesis 200 and/or the plurality of filaments 210 tends to resist axial and/or downstream migration, and when the peristaltic wave 91, the rhythmic forces of expansion and contraction caused by the peristaltic wave 91, and/or the radially compressive force ends or is removed, the endoprosthesis 200 may remain in its original position within the body lumen 90, as seen in FIG. 11C.

In some embodiments, an outer diameter of the endoprosthesis 200 may be generally uniform and/or generally constant along the length of the endoprosthesis 200 in the radially expanded configuration. In some embodiments, the endoprosthesis 200 may comprise a flared first end portion and/or a flared second end portion in the radially expanded configuration. In some embodiments, the outer diameter of the endoprosthesis 200 may be generally uniform and/or generally constant along the length of the endoprosthesis 200 except for the flared first end portion and/or the flared second end portion, where present, in the radially expanded configuration. In some embodiments, the outer diameter of the endoprosthesis 200 may be generally uniform and/or generally constant along the length of the endoprosthesis 200 from the flared first end portion to the flared second end portion, where present, in the radially expanded configuration. Other configurations are also contemplated.

In some embodiments an endoprosthesis may be made in the helical manner along only a portion of its length. For example, an endoprosthesis may be made in a conventional manner (e.g., consistent with the endoprosthesis 100) along a first portion and in the helical manner (e.g., consistent with the endoprosthesis 200) along a second portion. In another example, an endoprosthesis may be made in the conventional manner (e.g., consistent with the endoprosthesis 100) along a first end portion or the flared first end portion, in the helical manner (e.g., consistent with the endoprosthesis 200) along a medial portion, and in the conventional manner (e.g., consistent with the endoprosthesis 100) along a second end portion or the flared second end portion spaced apart from the first end portion or the flared first end portion by the medial portion. In yet another example, an endoprosthesis may be made in the helical manner (e.g., consistent with the endoprosthesis 200) along a first end portion or the flared first end portion, in the conventional manner (e.g., consistent with the endoprosthesis 100) along a medial portion, and in the helical manner (e.g., consistent with the endoprosthesis 200) along a second end portion or the flared second end portion spaced apart from the first end portion or the flared first end portion by the medial portion. Other configurations are also contemplated.

In some embodiments, the endoprosthesis 200 may comprise a polymeric covering (not shown) coupled thereto. In some embodiments, the polymeric covering may be fixedly attached to the endoprosthesis 200. In some embodiments, the polymeric covering may extend along an inner surface of the endoprosthesis 200. In some embodiments, the polymeric covering may extend along the outer surface of the endoprosthesis 200. In at least some embodiments, the endoprosthesis 200 may be embedded within the polymeric covering. Other configurations, including combinations thereof, are also contemplated. Some suitable but non-limiting examples of polymeric materials for the polymeric covering are discussed below.

In some embodiments, an endoprosthesis system may comprise the endoprosthesis 200 and a delivery device (not shown). In some embodiments, the delivery device may comprise an inner tubular member and an outer tubular member. The endoprosthesis 200 may be disposable within an annular space disposed between the inner tubular member and the outer tubular member (e.g., within a lumen of the outer tubular member and around and/or radially outward of the inner tubular member) in a collapsed delivery configuration. The inner tubular member and the outer tubular member may be axially movable relative to each other to load and/or deploy the endoprosthesis 200. In some embodiments, the inner tubular member may comprise a guidewire lumen extending therein. In some embodiments, the delivery device may comprise a distal tip disposed at a distalmost end of the inner tubular member. In at least some embodiments, the guidewire lumen may extend through the distal tip. In some alternative embodiments, the delivery device may comprise a crochet delivery device. Other configurations are also contemplated. In some embodiments, the endoprosthesis 200 may be configured to shift between the collapsed delivery configuration and an expanded deployed configuration. In some embodiments, the endoprosthesis 200 may be configured to self-expand from the collapsed delivery configuration to the expanded deployed configuration. In some embodiments, the endoprosthesis 200 may be balloon expandable from the collapsed delivery configuration to the expanded deployed configuration. Other configurations are also contemplated.

In some embodiments, the structure(s), configuration(s), and/or arrangement(s) described herein may be incorporated into other types of devices and/or methods. For example, filaments oriented at different angles relative to a central longitudinal axis may be incorporated within a reinforcing structure for a catheter (e.g., a guide catheter, a balloon catheter, etc.) and/or a composite shaft to provide desired torque and flexibility characteristics in accordance with the disclosure.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the endoprosthesis, the plurality of filaments, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly praraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50 A), polycarbonates, polyurethane silicone copolymers (for example, Elast-Eon® or ChronoSil®), biocompatible polymers, bioabsorbable polymers (for example, poly-l-lactic acid (PLLA), poly lactic-co-glycolic acid (PLGA), etc.), other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis having a length extending along a central longitudinal axis from a proximal end to a distal end, comprising:
a plurality of filaments interwoven to form a plurality of closed cells extending along the length of the endoprosthesis;
wherein each closed cell has corners defined by cross-over points formed by the plurality of filaments;
wherein each filament of the plurality of filaments forming a cross-over point of the plurality of cross-over points is oriented at a different angle relative to the central longitudinal axis.

2. The endoprosthesis of claim 1, wherein the plurality of filaments comprises at least a first filament extending in a first helical direction and a second filament extending in a second helical direction different from the first helical direction.

3. The endoprosthesis of claim 2, wherein the first filament is oriented at a first angle relative to the central longitudinal axis as viewed perpendicular to the central longitudinal axis at the cross-over point and the second filament is oriented at a second angle relative to the central longitudinal axis as viewed perpendicular to the central longitudinal axis at the cross-over point, the first angle being different from the second angle.

4. The endoprosthesis of claim 3, wherein the first angle and the second angle are interior angles within one closed cell of the plurality of closed cells.

5. The endoprosthesis of claim 3, wherein the cross-over point is a proximal cross-over point of a closed cell and an imaginary line extending through the proximal cross-over point of the closed cell and a distal cross-over point of the closed cell as viewed perpendicular to the central longitudinal axis at the proximal cross-over point of the closed cell is oriented at a third angle relative to the central longitudinal axis, the third angle being different from the first angle and the second angle.

6. The endoprosthesis of claim 3, wherein adjacent turns of the first filament are parallel to each other and adjacent turns of the second filament are parallel to each other.

7. The endoprosthesis of claim 1, wherein proximal and distal corners of each closed cell, as viewed in a direction parallel to the central longitudinal axis, are offset circumferentially from each other.

8. The endoprosthesis of claim 1, wherein opposing lateral corners of each closed cell, as viewed in a direction parallel to the central longitudinal axis, are offset longitudinally from each other.

9. The endoprosthesis of claim 1, further comprising at least one longitudinal reinforcing element interwoven within the plurality of filaments.

10. The endoprosthesis of claim 9, wherein the at least one longitudinal reinforcing element extends helically around the central longitudinal axis.

11. An endoprosthesis having a length extending along a central longitudinal axis from a proximal end to a distal end, comprising:
a plurality of filaments interwoven to form a plurality of closed cells extending along the length of the endoprosthesis;
wherein each closed cell has corners defined by cross-over points formed by the plurality of filaments, the cross-over points of each closed cell including a proximal cross-over point, a distal cross-over point, a first lateral cross-over point, and a second lateral cross-over point;
wherein the distal cross-over point of each closed cell is circumferentially offset from the proximal cross-over point of its respective closed cell.

12. The endoprosthesis of claim 11, wherein a first closed cell comprises a first proximal cross-over point and a first distal cross-over point and a second closed cell comprises a second proximal cross-over point and a second distal cross-over point;
wherein the second proximal cross-over point is the first distal cross-over point;
wherein the first proximal cross-over point, the first distal cross-over point, and the second distal cross-over point define an imaginary line oriented non-parallel to the central longitudinal axis.

13. The endoprosthesis of claim 12, wherein the first proximal cross-over point, the first distal cross-over point, and the second distal cross-over point are aligned helically around the central longitudinal axis.

14. The endoprosthesis of claim 11, wherein the plurality of filaments is configured to resist axial elongation of the endoprosthesis when a radially compressive force is applied to the endoprosthesis.

15. A method of manufacturing an endoprosthesis, comprising:
securing a plurality of filaments extending from a braiding machine to a mandrel such that the plurality of filaments forms a braid tent oriented at an intersecting angle with the mandrel; and
interweaving the plurality of filaments around the mandrel to form a plurality of closed cells having corners defined by cross-over points formed by the plurality of filaments while rotating the mandrel around a central longitudinal axis of the mandrel.

16. The method of claim 15, wherein each filament of the plurality of filaments forming a cross-over point is oriented at a different angle relative to the central longitudinal axis as viewed perpendicular to the central longitudinal axis at the cross-over point.

17. The method of claim 16, wherein a first filament of the plurality of filaments extending in a first helical direction around the central longitudinal axis and a second filament of the plurality of filaments extending in a second helical direction around the central longitudinal axis intersect at the cross-over point such that the first filament is oriented at a first angle relative to the central longitudinal axis as viewed perpendicular to the central longitudinal axis at the cross-over point, and the second filament is oriented at a second angle relative to the central longitudinal axis as viewed perpendicular to the central longitudinal axis at the cross-over point, the first angle being different from the second angle.

18. The method of claim 15, wherein interweaving the plurality of filaments around the mandrel comprises simultaneously rotating the mandrel relative to the braiding machine while translating the mandrel longitudinally relative to the braiding machine.

19. The method of claim 18, wherein a first puller mechanism is disposed on a first side of the mandrel and is oriented at a first angle to the mandrel, and a second puller mechanism is disposed on a second side of the mandrel opposite the first side relative to the central longitudinal axis and is oriented at a second angle to the mandrel;
wherein the first puller mechanism and the second puller mechanism cooperate to rotate the mandrel relative to the braiding machine while translating the mandrel longitudinally relative to the braiding machine.

20. The method of claim 15, wherein a filament guide spaced apart from the braiding machine within the braid tent is configured to redirect the plurality of filaments extending from the braiding machine to the mandrel such that the intersecting angle with the mandrel is between 70 degrees and 90 degrees.
